SEQUENCE LISTING (6 sequences)

SEQ ID NO: 1 - DNA, 28 bp, Artificial Sequence
  primer oligonucleotide
agggagttttttttagggaatagaggga

SEQ ID NO: 2 - DNA, 28 bp, Artificial Sequence
  primer oligonucleotide
taatcccaaaacctctccactacaacaa

SEQ ID NO: 3 - DNA, 18 bp, Artificial Sequence
  primer oligonucleotide
ttccacttaatcgctccc

SEQ ID NO: 4 - DNA, 18 bp, Artificial Sequence
  primer oligonucleotide
agagttttcgtagttttt

SEQ ID NO: 5 - DNA, 18 bp, Artificial Sequence
  primer oligonucleotide
ttccacttaatcactccc

SEQ ID NO: 6 - DNA, 18 bp, Artificial Sequence
  primer oligonucleotide
agagtttttgtagttttt

(19) United States
(12) Patent Application Publication
(10) Pub. No.: US 2004/0132026 A1
(43) Pub. Date: Jul. 8, 2004

(54) Method for determining the age of individuals

(76) Inventor: Alexander Olek, Berlin (DE)

Correspondence Address: KRIEGSMAN & KRIEGSMAN, 665 FRANKLIN STREET, FRAMINGHAM, MA, 01702, US

(21) Appl. No.: 10/343,740

(22) PCT Filed: Aug. 2, 2001
(86) PCT No.: PCT/DE01/02916

(30) Foreign Application Priority Data
Aug. 2, 2000 (DE) 100-38-733.0

(51) Int. Cl. C12Q 1/68; G06F 19/00; G01N 33/48; G01N 33/50

(52) U.S. Cl. 435/6; 702/20

(57) ABSTRACT

A method is described for the determination of the age of individuals based on sperm samples, which is characterized in that age information is determined by analysis of DNA methylation patterns of the DNA contained in sperm samples.

The invention concerns a method for the age determination of individuals.

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, the age of an individual is expressed by a modified methylation pattern of individual genes or of the genome.

Increasingly more frequently, when following up a crime at the crime scene or in the case of biological material found as trace material from the victim, an investigation is conducted by means of DNA analysis and the material is compared with other DNA materials.

Taking into consideration the worldwide research in the field of genome analysis, in the meantime, concrete information can be obtained via genetic dispositions that have information value with regard to content. The storage and utilization of investigative results obtained by DNA analysis in databases for purposes of criminal record-keeping is of utmost importance.

Since 1985, the typing of biological material has been one of the most important methods for identification of individuals in forensic medicine and in criminal investigations (Jeffreys A J, Wilson V, Thein S L. Hypervariable ‘minisatellite’ regions in human DNA. Nature. 1985 Mar. 7-13;314(6006):67-73; Benecke M. DNA typing in forensic medicine and in criminal investigations: a current survey. Naturwissenschaften 1997 May;84(5):181-8). In addition to DNA fingerprinting for identification of persons, there is also the possibility of determining the age of a man based on the methylation pattern of his sperm.

Sex-specific and sequence-specific methylation patterns of mammalian DNA are established duing gametogenesis. It is assumed that they participate decisively in genomic imprinting and in development-controlled gene regulation. Investigations, which are concerned with the expression of enzymes that particpate in DNA methylation, show that such an enzyme is clearly regulated by developmental biology during spermatogenesis at the stage of mRNA, protein and enzyme activity (Benoit G, Trasler J M. Developmental expression of DNA methyltransferase messenger ribonucleic acid, protein, and enzyme activity in the mouse testis. Biol Reprod. 1994 June;50(6):1312-9). Changes in the 5-methyldeoxycytidine pattern of DNA influence the gene expression of specific mammalian genes with respect to development, differentiation, carcinogenesis and aging. The detection of DNA methylation in the promoter region, a process which normally suppresses transcription activity, is an important investigative criterion in relation to changes in molecular expression for disorders caused by age (Nagane Y, Utsugisawa K, Tohgi H. PCR amplification in bisulfite methylcytosine mapping in the GC-rich promoter region of amyloid precursor protein gene in autopsy human brain. Brain Res Brain Res Protoc. 2000 April;5(2):167-71.).

The methylation state of genes which contain CpG-rich regions (CpG islands), has been investigated in human sperm, fetal and adult tissues (Ghazi H, Gonzales F A, Jones P A. Methylation of CpG-island-containing genes in human sperm, fetal and adult tissues. Gene. 1992 May 15;114(2): 203-10). Changes in methylation during various stages of development were investigated for different human genes. In one of these genes, which codes for insulin, it was detected that the gene was abundantly methylated in sperm, that it was less methylated in fetal tissue independent of expression, and an increased methylation was present in adult tissue. In more recent investigations, which are concerned with the special methylation patterns of factor VIII DNA which was isolated from sperm, it could be shown that there are not only differences in the mutation frequency among CpG sites, but also between two ethnic groups. The results further clarify that different CpG sites vary in their methylation pattern not only within the same individual, but also between different individuals (Millar D S, Krawczak M, Cooper D N. Variation of site-specific methylation patterns in the factor VIII (F8C) gene in human sperm DNA. Hum Genet. 1998 August; 103(2):228-33.)

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now completely utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

With few exceptions (e. g. Zechnigk, M. et al., Eur. J. Hum. Gen. 1997, 5, 94-98) the bisulfite technique has been previously applied only in research. However, short, specific segments of a known gene have always been amplified according to a bisulfite treatment and either competely sequenced (Olek, A. and Walter, J., Nat. Genet. 1997,17, 275-276) or individual cytosine position are detected by a “primer extension reaction” (Gonzalgo, M. L. and Jones, P. A., Nucl. Acids Res. 1997, 25, 2529-2531, WO-A 95 00669) or an enzyme step (Xiong, Z. and Laird, P. W., Nucl. Acids Res. 1997, 25, 2532-2534). Detection has also been described by hybridization (Olek et al., WO A 99 28498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97 46705 and WO A-95 15373.

An overview of the state of the art in oligomer array production can be derived also from a special issue of Nature Genetics which appeared in January 1999 (Nature Genetics Supplement, Volume 21, January 1999), the literature cited therein and U.S. Pat. No. 5,994,065 on methods for the production of solid supports for target molecules such as oligonucleotides in the case of reduced nonspecific background signal.

The object of the invention is to provide a method which permits the analysis of the age of individuals by means of molecular biological techniques. DNA methylation patterns will be used for this purpose. In particular, the problem is to be solved in such a way that these special methylation patterns must not depend on membership in a specific ethnic group or on, e.g., environmental influences, which otherwise falsify the age determination.

The present invention thus describes a method for the detection of the methylation state of genomic DNA with the objective of determining the age of an individual.

The task is solved by a method for the determination of the age of individuals, whereby the DNA methylation pattern is analyzed in a DNA sample of the individual.

It is most particularly preferred that the DNA sample is a sperm sample.

According to the invention, a method is preferred in which:

a) Specific CpG dinucleotides are analyzed in genomic DNA for their degree of methylation and this is done separately for different subjects of different age;

b) The degree of methylation of these CpG dinucleotides is correlated with the age of the subject and this information is stored in a database;

c) The age of an individual is determined on the basis of the methylation analysis of CpG dinucleotides of a DNA sample of the individual by comparing the analytical results with the database information.

According to the invention, a method is also preferred for determining the age of individuals in which the following process steps are conducted:

a) The DNA contained in the samples is chemically treated in such a way that 5-methylcytosine and cytosine react differently and cytosine is selectively converted into a base with a base-pairing behavior that is different from that of cytosine.

b) Portions of the base sequence of the DNA segments are determined;

c) The obtained bases sequences are compared with a database, which correlates sequences with the age of individuals.

A method is further preferred according to the invention, in which

a) The DNA contained in the samples is chemically treated in such a way that 5-methylcytosine and cytosine react differently and cytosine is selectively converted into a base with a base-pairing behavior that is different from that of cytosine;

b) The fragments of the DNA treated in this way are amplified;

c) The fragments are hybridized to a set of oligomers;

d) The unhybridized fragments are removed;

e) The hybridized fragments are analyzed and the result is compared with a database, which correlates the hybridization pattern with the age of individuals.

A method is further preferred according to the invention, in which

a) The DNA contained in the samples is chemically treated in such a way that 5-methylcytosine and cytosine react differently and cytosine is selectively converted into a base with a base-pairing behavior that is different from that of cytosine;

b) The fragments of the DNA treated in this way are amplified;

c) The fragments are hybridized to a set of primer oligonucleotides;

d) The primers are extended in a sequence-specific reaction;

e) The extension products are analyzed and the result is compared with a database, which correlates the analytical results with the age of individuals.

In the method according to the invention, it is most particularly preferred that the analysis is conducted with oligonucleotide arrays.

In the method according to the invention, it is most particularly preferred also that the analysis is conducted by means of mass spectrometry.

A method according to the invention is thus described for the determination of the age of individuals, preferably based on sperm samples.

The information for the determination of the age of individuals is obtained by a method for the analysis of DNA methylation patterns in a DNA sample.

In a particularly preferred embodiment of the method for the determination of the age of individuals, sperm samples are investigated, wherein the age information is obtained by analysis of DNA methylation patterns of the sample DNA contained in the sperm samples.

The following embodiments are preferred for this determination of the age of individuals:

In a preferred embodiment, defined CpG dinucleotides in genomic DNA are investigated for their extent of methylation and this is done separately for different subjects of different age. The degree of methylation of the CpG dinucleotides is correlated with the age of the subject and this information is stored in a database. The age of an individual is determined on the basis of the methylation analysis of the CpG dinucleotides of a DNA sample of the individual by comparing the analytical results with the database information.

In another preferred embodiment, the DNA contained in the samples is chemically treated in such a way that 5-methylcytosine and cytosine react differently and cytosine is selectively converted into a base with a base-pairing behavior that is different from that of cytosine. Bisulfite is preferably used, so that an addition takes place at the unmethylated cytosine bases. The subsequent alkaline hydrolysis then leads to the conversion of unmethylated cytosine nucleobases to uracil. Then portions of the base sequence of the DNA segments are determined and the obtained base sequences are compared with a database that correlates the sequences with the age of individuals.

In another preferred embodiment, the DNA contained in the samples is chemically treated in such a way that 5-methylcytosine and cytosine react differently and cytosine is selectively converted into a base with a base-pairing behavior that is different from that of cytosine. Then fragments of the pretreated DNA are amplified with the use of a heat-stable polymerase and at least one primer is preferably amplified with the polymerase [chain] reaction (PCR). Various defined amplifications are conducted in one reaction vessel. This is preferably a so-called multiplex PCR, in which different primers generate defined fragments. In another variant of the method, the primers each amplify several fragments in a targeted and reproducible manner. In a particularly preferred variant of the method, the ampification occurs by the extension of primers, which are bound to a solid phase. A multiplex PCR can be conducted in another sense, in that different primers are bound to different, defined sites of a solid phase. The solid phase is usually planar, whereby the different oligonucleotide sequences are arranged in the form of a rectangular or hexagonal grid. As a consequence, the different amplified products are also arranged on the solid phase in the form of a rectangular or hexagonal grid. As already described above, in this case, several amplified products are generated directly on the solid phase. The solid-phase surface is preferably comprised of silicon, glass, polystyrene, aluminum, steel, iron, copper, nickel, silver, or gold. The fragments of the amplified genomic DNA are hybridized to a set of oligomers (primers) with the formation of a duplex. The unhybridized fragments are then removed. Subsequently, the hybridized fragments are analyzed and the result is compared with a database, which correlates the hybridization pattern with the age of individuals.

In a particularly preferred embodiment, the DNA contained in the samples is chemically treated in such a way that 5-methylcytosine and cytosine react differently and cytosine is selectively converted into a base with a base-pairing behavior that is different from that of cytosine. Then fragments of the pretreated DNA are amplified with the use of a heat-stable polymerase and at least one primer is preferably amplified with the polymerase [chain] reaction (PCR). Various defined amplifications are conducted in one reaction vessel. This is preferably a so-called multiplex PCR, in which different primers generate defined fragments. In another variant of the method, the primers each amplify several fragments in a targeted and reproducible manner. In a particularly preferred variant of the method, the ampification occurs by the extension of primers, which are bound to a solid phase. A multiplex PCR can be conducted in another sense, in that different primers are bound to different, defined sites of a solid phase. The solid phase is usually planar, whereby the different oligonucleotide sequences are arranged in the form of a rectangular or hexagonal grid. As a consequence, the different amplified products are also arranged on the solid phase in the form of a rectangular or hexagonal grid. As already described above, in this case, several amplified products are generated directly on the solid phase. The solid-phase surface is preferably comprised of silicon, glass, polystyrene, aluminum, steel, iron, copper, nickel, silver, or gold. The fragments are then hybridized to a set of primer oligonucleotides and the primers are extended in a sequence-specific reaction with a heat-stable polymerase. Preferably, at least one nucleotide bears a detectable label. The type of extension thus depends on the methylation state of the respective cytosine in the genomic DNA sample. Subsequently, the extension producs are analyzed and the result is compared with a database, which correlates the hybridization pattern with the age of individuals.

The analysis of the DNA methylation patterns of the above-given embodiments is conducted in a particularly preferred manner by means of mass spectrometry.

The following examples explain the invention:

### EXAMPLE 1

### Description of the PCR

The PCR reaction for the individual gene was conducted by means of a thermocycler (Epperdorf GmbH) with the use of 10 ng of bisulfite-treated DNA, 12.5 pmol of each primer, 1 mM of each dNTP, 1.5 mM MgCl₂ und 1 U of HotstartTaq (Qiagen AG). The other conditions corresponded to those which were recommended by the manufacturer of the Taq polymerase. Individual genes were amplified by PCR by conducting a first denaturation step for 20 min. at 95° C., followed by 45 cycles (60 sec. at 95° C, 45 sec. at 55° C., 75 sec. at 72° C.) and a subsequent elongation of 10 min. at 72° C.

### EXAMPLE 2

### The Following Examples Refer to a Fragment of Exon 40 of the FVIII Gene, in Which Specific CG Positions were to be Analyzed for Methylation

In the first step, a genomic sequence is treated with the use of bisulfite (hydrogen sulfite, disulfite) in such a way that all of the cytosines not methylated at the 5-position of the base are modified such that a base that is different with respect to its base-pairing behavior is substituted, whereas the cytosines that are methylated in the 5-position remain unchanged.

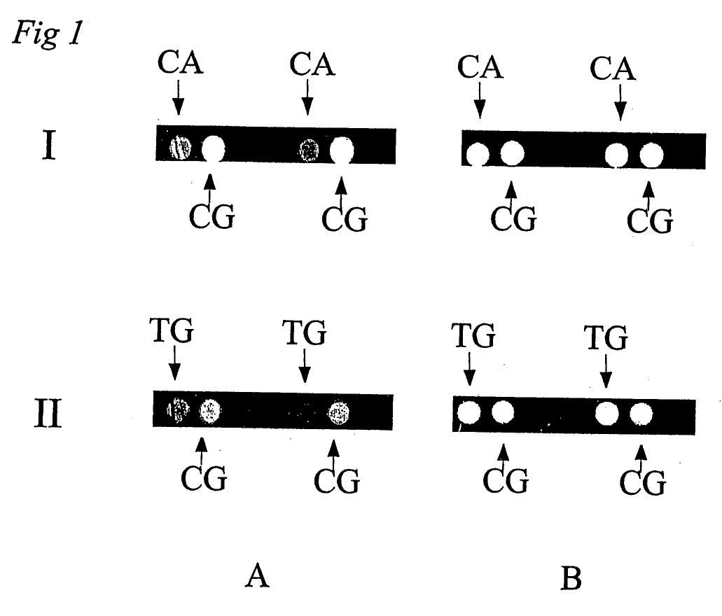

If bisulfite solution is used for the reaction, then an addition occurs on the unmethylated cytosine bases. Also, a denaturing reagent or solvent as well as a radical trap must be present. A subsequent alkaline hydrolysis then brings about the conversion of the unmethylated cytosine bases to uracil. The chemically converted DNA is then used for the detection of methylated cytosines. In the second step of the method, the treated DNA sample is diluted with water or an aqueous solution. Preferably, the DNA is then desulfonated. In the third step of the method, the DNA sample is amplified in a polymerase chain reaction, preferably with the use of a heat-stable DNA polymerase, as described in Example 1. In the present case, cytosines of exon 11 of the FVIII gene are analyzed. For this purpose a defined fragment with a length of 561 bp is amplified with the specific primer oligonucleotides AGGGAGTTTTTTTTAGGGMTAGAGGGA and TAATCCCAAAACCTCTCCACTACAACAA. The amplified product serves as the sample, which is hybridized to oligonucleotides which were previously bound to a solid phase, and a duplex structure is formed, for example, TTCCACTTAATCGCTCCC (the CG of this oligonucleotide is shown in FIG. 1, I) or AGAGTTTTCGTAGTTTTT (the CG of this oligonucleotide is shown in FIG. 1, II), whereby the cytosine to be detected is found at position 30 or at position 500 of the amplified product. The detection of the hybridization product is based on Cy5-fluorescently-labeled primer oligonucleotides, which have been used for the amplification. A hybridization reaction of the amplified DNA with the oligonucleotide occurs only if a methylated cytosine was present at this site in the bisulfite-treated DNA, as is shown in FIG. 1. Consequently, the methylation state of the specific cytosine to be analyzed is derived from the hybridization product.

In order to analye the unmethylated state, the oligonucleotides TTCCACTTAATCACTCCC (the CA of this nucleotide is shown in FIG. 1, I) or AGAGTTTTTGTAGTTTTT (the TG of this oligonucleotide is shown in FIG. 1, II) are used. These oligonucleotides have a thymine base instead of a cytosine at the positions to be analyzed. Therefore, the hybridization reaction occurs only if an unmethylated cytosine was present at the position to be analyzed, as is shown in FIG. 1. It is shown for two different oligonucleotides that the CpG positions to be analyzed have a different degree of methylation for a 41-year old subject (FIG. 1, A) compared with a 23-year old subject (FIG. 1, B). The signal intensities of the two oligonucleotides for the methylated state, shown by the oligonucleotides containing the CG of the 41-year-old subject (FIG. 1, A) are higher than the intensities of the oligonucleotide of this patient containing CA, representing the unmethylated state. In contrast to this, the signal intensities of the oligonucleotides representing the methylated state and those of the oligonucleotides representing the methylated state are nearly the same for the 23-year-old subject (FIG. 1, B). *sic; unmethylated?—Trans. Note.

### DESCRIPTION OF FIG. 1

FIG. 1 shows the hybridization of fluorescently-labeled amplified products on surface-bound oligonucleotides for various oligonucleotides (as shown in FIG. 1 repeated twice for each oligonucleotide). Oligonucleotide samples A originate from the 41-year-old subject and samples B originate from the 23-year-old subject. The fluorescence on one spot, characterized by an arrow, shows the hybridization of the amplified product on the oligonucleotide. Hybridization to a CG-containing oligonucleotide characterizes a methylation at the analyzed cytosine position, while hybridization to an oligonucleotide containing CA or TG characterizes an unmethylated cytosine at the cytosine position to be analyzed.

1. A method for the determination of the age of individuals, hereby characterized in that the DNA methylation patterns are analyzed in a DNA sample of the individual.

2. The method for the determination of the age of individuals according to claim 1, further characterized in that the DNA sample is a sperm sample.

3. The method for the determination of the age of individuals according to claim 1 or 2, further characterized in that
a) Specific CpG dinucleotides in genomic DNA are analyzed for their degree of methylation and this is done separately for different subjects of different age;
b) The degree of methylation of these CpG dinucleotides is correlated with the age of the subject and this information is stored in a database;
c) The age of an individual is determined on the basis of the methylation analysis of the CpG dinucleotides of a DNA sample of the individual by comparing the analytical results with the database information.

4. The method for the determination of the age of individuals according to one of the preceding claims, further characterized in that the following method steps are conducted:
a) The DNA contained in the samples is chemically treated in such a way that 5-methylcytosine and cytosine react differently and cytosine is selectively converted into a base with a base-pairing behavior that is different from that of cytosine.
b) Portions of the base sequence of the DNA segments are determined;
c) The obtained bases sequences are compared with a database, which correlates sequences with the age of individuals.

5. The method for the determination of the age of individuals on the basis of DNA samples according to one of the preceding claims, further characterized in that
a) The DNA contained in the samples is chemically treated in such a way that 5-methylcytosine and cytosine react differently and cytosine is selectively converted into a base with a base-pairing behavior that is different from that of cytosine;
b) The fragments of the DNA treated in this way are amplified;
c) The fragments hybridize to a set of oligomers;
d) the unhybridized fragments are removed;
e) The hybridized fragments are analyzed and the result is compared with a database, which correlates the hybridization pattern with the age of individuals.

6. The method for the determination of the age of individuals on the basis of DNA samples according to one of claims 1 to 4, further characterized in that
a) The DNA contained in the samples is chemically treated in such a way that 5-methylcytosine and cytosine react differently and cytosine is selectively converted into a base with a base-pairing behavior that is different from that of cytosine;
b) The fragments of the DNA treated in this way are amplified;
c) The fragments are hybridized to a set of primer oligonucleotides;
d) The primers are extended in a sequence-specific reaction;
e) The extension products are analyzed and the result is compared with a database, which correlates the analytical results with the age of individuals.

7. The method according to one of the preceding claims, further characterized in that the analysis is conducted with oligonucleotide arrays.

8. The method according to one of claims 1 to 7, further characterized in that the analysis is conducted by means of mass spectrometry.
